(19) United States
(12) Patent Application Publication
(10) Pub. No.: US 2020/0390666 A1
(43) Pub. Date: Dec. 17, 2020

(54) RINSE-FREE SHAMPOO COMPOSITION

(71) Applicant: Church & Dwight Co., Inc., Princeton, NJ (US)

(72) Inventor: Alexander John Richardson, Gillingham (GB)

(73) Assignee: Church & Dwight Co., Inc., Princeton, NJ (US)

(21) Appl. No.: 16/863,270

(22) Filed: Apr. 30, 2020

Related U.S. Application Data
(60) Provisional application No. 62/841,451, filed on May 1, 2019.

(51) Int. Cl. A61K 8/04; A61K 8/34; A61K 8/894; A61K 8/41; A61K 8/92; A61K 8/73; A61K 8/31; A61Q 5/02

(52) U.S. Cl. CPC A61K 8/046; A61K 8/34; A61K 8/347; A61K 8/894; A61K 8/416; A61K 8/922; A61K 2800/244; A61K 8/31; A61Q 5/02; A61K 2800/87; A61K 2800/52; A61K 2800/48; A61K 8/732

(57) ABSTRACT

The present disclosure provides rinse-free shampoo compositions effective for cleaning hair without leaving behind a residue. The rinse-free shampoo composition includes an aqueous liquid phase, wherein the liquid phase is about 60-70% of the total weight of the rinse-free shampoo composition; a solid phase comprising a starch thickener, wherein the solid phase is about 4-15% of the total weight of the rinse-free shampoo composition; and a gas phase comprising at least one propellant, wherein the gas phase is about 20-35% of the total weight of the rinse-free shampoo composition.

### CROSS-REFERENCE TO RELATED APPLICATIONS

The present application claims priority to U.S. Provisional Application No. 62/841,451, filed on May 1, 2019, which is herein incorporated by reference in its entirety.

### FIELD OF THE INVENTION

The present disclosure relates to compositions for cleaning hair, and more particularly to a rinse-free shampoo composition.

### BACKGROUND

This disclosure relates to a natural, rinse-free shampoo and conditioning composition capable of imparting superior cleaning and conditioning ability on hair.

Dry hair cleaning compositions for the rapid cleansing of hair are well known. These known compositions can be used on dry hair in order to freshen up the hair and/or to remove excess oils and other undesirables found on dirty hair. These known compositions are particularly useful when time is short and one cannot wash the hair using wet shampoo.

Dry shampoo can be defined as a shampoo in a powder form, used without the addition of water. The most common conventional dry shampoo format is a shampoo in the form of an aerosol. The aerosol is sprayed into the roots of the hair, massaged in and combed and this process is done very quickly thanks to the no rinse formulation. Derivatives of the aerosol have been seen as shakers, but still use the same type of powder seen in the aerosol dry shampoos.

The powders used in dry shampoos are typically a starch or starch derivative and although different starches offer different properties, they all come to the same end result: quick hair cleaning. Although conventional dry (rinse-free) shampoos can be effective at removing oils from the treated hair, the conventional powders are never fully brushed out of the hair and is instead left in the treated hair, which can result in the hair not feeling fully clean. Furthermore, conventional dry shampoo powders can leaver a residue (e.g., white, off-white, or other color depending on the powder color) in the treated hair, which consumers find undesirable.

Alternative solutions have been proposed to deliver a better cleaning experience using an aqueous formulation with a foaming pump. The operator will use a manual pump action to generate foam which is applied to the hand, which is then applied to the hair. For example, Zerreau™ Towel Off Shampoo (http://waterlessltd.co.uk/), Axe™ Reset Waterless Foam Shampoo (https://www.unilever.com/), and TRESemme™ Fresh Start Waterless Foam Shampoo (https://www.unilever.com/), are exemplary aqueous formulations with a foaming pump. These products use standard detergents (primary and secondary surfactants) to clean the hair to remove the oils and dirt on the hair. These products give a feeling of cleanliness that is similar to conventional shampooing, i.e., in the shower. However, by the nature of the density of foam generated and the required rate of application, these and other similar products leave the hair wet after use and a towel/absorbent cloth is required to dry the hair. This means the user is required to have access to a removal device (towel/cloth/etc) and the action of removing the foam takes time and overall is not an efficient way to achieve clean, dry hair quickly.

Improved versions of the aqueous formulation with a foaming pump have been seen on the market in the form of mousse aerosols with an upright delivery. Examples of these products are Ouai™ Dry Shampoo Foam (https://theouai.co.uk/products/dry-shampoo-foam), Umberto Giannini™ Shower in a bottle (https://umbertogiannini.com/shop/products/shower-in-a-bottle-magic-foam-volume-cleanser), and Amika™—Dry Shampoo Foam (https://loveamika.com/products/phantom-p-hydrating-dry-shampoo-foam). These products use standard secondary surfactants (e.g., Cocamidopropyl Betaine) or foam boosters (e.g., disodium cocoamphodipropionate) to generate a foam to be applied to the hair to remove oils. These products are designed to refresh the hair. However, due to the format of an upright mousse application the product is applied to the hair in a very dense, very wet foam that requires a lot of energy to make the hair dry after use. This means that the format is not quick and easy to use.

Accordingly, there is still a desire and a need to provide a dry shampoo composition that is suitable for both cleaning dry hair and has the perceived cleaning performance of the upright mousse aerosols, but does not require a lot of energy to dry the hair. In particular, there is a need to provide an aqueous foaming product that has the perceived cleaning performance of the pump foam, but leaves the dry hair of a dry shampoo, whilst leaving no visible residue. Further, it is desirable to provide a dry shampoo that does not require a secondary component or the use of water, with the exception of an optional brush.

### SUMMARY OF THE INVENTION

In one aspect of the present disclosure, a rinse-free shampoo composition is provided, the rinse-free shampoo composition comprising an aqueous liquid phase, wherein the liquid phase is about 60-70% of the total weight of the rinse-free shampoo composition; a solid phase comprising a starch thickener, wherein the solid phase is about 4-15% of the total weight of the rinse-free shampoo composition; and a gas phase comprising at least one propellant, wherein the gas phase is about 20-35 of the total weight of the rinse-free shampoo composition.

In various embodiments, the liquid phase comprises an alcohol in the amount of about 1-15%, based on the total weight of the rinse-free shampoo composition. The alcohol can be ethanol, for example. In some embodiments, wherein the liquid phase comprises stabilizer in the amount of about 0.01-1%, based on the total weight of the rinse-free shampoo composition. The stabilizer can be phenoxyethanol, for example. In various embodiments, the liquid phase comprises a conditioning agent in the amount of about 0.01-5%, based on the total weight of the rinse-free shampoo composition. The conditioning agent can be PEG-8 dimethicone, for example. In some embodiments, the liquid phase comprises an amphoteric surfactant in the amount of about 0.01-3%, based on the total weight of the rinse-free shampoo composition. The amphoteric surfactant can be cocamidopropyl betaine, for example. In certain embodiments, the liquid phase further comprises at least one of a fragrance, extracts, and preservatives. In various embodiments, the liquid phase has a viscosity of about 1-10 centipoise.

In some embodiments, the starch thickener is distarch phosphate, for example. In certain embodiments, the at least one propellant comprises isobutane, propane, butane, or a combination thereof.

The rinse-free shampoo compositions described herein can be provided in a container from which the rinse-free shampoo composition is dispensable in the form of a foam. In various embodiments, the foam has a density of about 0.1-0.2 g/cm³.

A method of cleaning hair is also provided herein, wherein the method comprises applying the rinse-free shampoo composition described herein onto the hair of a human or animal. The rinse-free shampoo can be delivered at a rate of 0.75-3.0 g/s to the hair. Any device known in the art for storing aerosol and foam/mousse compositions can be used to contain the rinse-free shampoo compositions described herein.

The invention includes, without limitation, the following embodiments.

Embodiment 1: A rinse-free shampoo composition, comprising: an aqueous liquid phase, wherein the liquid phase is about 60-70% of the total weight of the rinse-free shampoo composition; a solid phase comprising a starch thickener, wherein the solid phase is about 4-15% of the total weight of the rinse-free shampoo composition; and a gas phase comprising at least one propellant, wherein the gas phase is about 20-35% of the total weight of the rinse-free shampoo composition.

Embodiment 2: The rinse-free shampoo composition of Embodiment 1, wherein the liquid phase comprises an alcohol in the amount of about 1-15%, based on the total weight of the rinse-free shampoo composition.

Embodiment 3: The rinse-free shampoo composition of Embodiment 2, wherein the alcohol is ethanol.

Embodiment 4: The rinse-free shampoo composition of any of Embodiments 1-3, wherein the liquid phase comprises stabilizer in the amount of about 0.01-1%, based on the total weight of the rinse-free shampoo composition.

Embodiment 5: The rinse-free shampoo composition of Embodiments 4, wherein the stabilizer is phenoxyethanol.

Embodiment 6: The rinse-free shampoo composition of any of Embodiments 1-5, wherein the liquid phase comprises a conditioning agent in the amount of about 0.01-5%, based on the total weight of the rinse-free shampoo composition.

Embodiment 7: The rinse-free shampoo composition of Embodiment 6, wherein the conditioning agent is PEG-8 dimethicone.

Embodiment 8: The rinse-free shampoo composition of any of Embodiments 1-7, wherein the liquid phase comprises an amphoteric surfactant in the amount of about 0.01-3%, based on the total weight of the rinse-free shampoo composition.

Embodiment 9: The rinse-free shampoo composition of Embodiment 8, wherein the amphoteric surfactant is cocamidopropyl betaine.

Embodiment 10: The rinse-free shampoo composition of any of Embodiments 1-9, further comprising at least one of a fragrance, extracts, and preservatives.

Embodiment 11: The rinse-free shampoo composition of any of Embodiments 1-10, wherein the liquid phase further comprises a cooling agent present in an amount of about 0.001 to about 1 weight percent, based on the total weight of the rinse-free shampoo composition.

Embodiment 12: The rinse-free shampoo composition of Embodiment 11, wherein the cooling agent is menthol.

Embodiment 13: The rinse-free shampoo composition of any of Embodiments 1-12, wherein the liquid phase further comprises PEG-40 hydrogenated castor oil in an amount of about 0.01 to about 1 weight percent, based on the total weight of the rinse-free shampoo composition.

Embodiment 14: The rinse-free shampoo composition of any of Embodiments 1-13, wherein the starch thickener is distarch phosphate.

Embodiment 15: The rinse-free shampoo composition of any of Embodiments 1-14, wherein the at least one propellant comprises isobutane, propane, butane, or a combination thereof.

Embodiment 16: The rinse-free shampoo composition of any of Embodiments 1-15, wherein the liquid phase has a viscosity of about 1-10 centipoise.

Embodiment 17: The rinse-free shampoo composition of any of Embodiments 1-16, wherein the rinse-free shampoo composition is provided in a container from which the rinse-free shampoo composition is dispensable in the form of a foam.

Embodiment 18: The rinse-free shampoo composition of Embodiment 17, wherein the foam has a density of about 0.1-0.2 g/cm³.

Embodiment 19: A method of cleaning hair comprising applying the rinse-free shampoo composition of any of Embodiments 1-18 onto the hair of a human or animal.

Embodiment 20: The method of Embodiment 19, wherein the rinse-free shampoo is delivered at a rate of 0.75-3.0 g/s to the hair.

Embodiment 21: A device containing the rinse-free shampoo composition of any of Embodiments 1-16.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### BRIEF DESCRIPTION OF THE DRAWINGS

FIG. 1 shows box and whisker plots of maximum load (gf) grouped by treatment;

FIG. 2 shows box and whisker plots of technical shine of treated hair tresses;

FIG. 3A is a FTIR spectra for virgin hair with no sebum added;

FIG. 3B is a FTIR spectra for hair after sebum application;

FIG. 4A is a FTIR spectra for hair samples after sebum application;

FIG. 4B is a FTIR spectra for hair samples after two times application of a dry shampoo composition according to the present disclosure; and

FIG. 5 shows box and whisker plots of number of broken fibers after 2,000 brush strokes.

### DETAILED DESCRIPTION OF THE INVENTION

The present disclosure now will be described more fully hereinafter with reference to the accompanying drawings. The disclosure may be embodied in many different forms and should not be construed as limited to the embodiments set forth herein; rather, these embodiments are provided so that this disclosure will satisfy applicable legal requirements. Like numbers refer to like elements throughout. As used in this specification and the claims, the singular forms “a,” “an,” and “the” include plural references unless the context clearly dictates otherwise.

In one aspect of the present disclosure, a rinse-free shampoo composition is provided which can be topically applied to hair. The term “rinse-free shampoo composition” is understood to relate to a product that is effective to cleanse hair without any necessity for rinsing with water following application. Wet shampoo compositions are understood to be applied to wet hair, lathered, and then rinsed away with added water. A rinse-free shampoo composition, however, can be applied to hair and then optionally be brushed through the hair with the fingers or a utensil (e.g., a comb or brush) to effect the cleansing effect. No rinsing with water is required. A certain amount of liquid components may be included in a rinse-free shampoo composition, as discussed in more detail below.

As described herein, it was surprisingly found that it is possible to propel a modified detergent formulation, as disclosed herein, in such a way that a rich foam is generated that feels wet when it is first applied to treated hair, thereby giving the user a shower-like feeling. However, almost immediately after massaging the modified detergent formulations disclosed herein into the hair, the hair is left in a touch-dry state leaving no visible residue. Without being limited by theory, the energy used to massage the detergent formulation into the hair can contribute to leaving the treated hair in a touch-dry state.

Preferably, a rinse-free shampoo composition as disclosed herein comprises a liquid phase having a water base and a secondary surfactant, and a solid phase having a starch base, and a gas phase including a propellant for the delivery of the aqueous solution. In various embodiments, the liquid phase comprises about 60-70% of the total weight of the rinse-free shampoo composition. In various embodiments, the solid phase comprises about 1-10%, or about 4-8% of the total weight of the rinse-free shampoo composition. In various embodiments, the gas or propellant phase comprises about 20-35% of the total weight of the rinse-free shampoo composition.

### Liquid Phase

In various embodiments, the liquid phase of the rinse-free shampoo composition can include an alcohol which can be useful in lowering the surface tension of the foam dispensed to the hair such that the foam breaks down at a desirable rate. In various embodiments, the alcohol can be present in an amount of about 1-15%, about 2-10%, or about 5-8%, based on the total weight of the rinse-free shampoo composition (i.e., including all three phases). In certain embodiments, the weight percentage of alcohol(s) in the rinse-free shampoo composition, based on the total weight of the rinse-free shampoo composition, can be at least about 0.1%, at least about 3%, or at least about 5%, preferably with an upper limit of about 15% by weight. In certain embodiments, the alcohol can be ethanol. However, any alcohol known in the art that can assist with altering the surface tension of the dispersed foam can be used.

In certain embodiments, the rinse-free shampoo composition can further include a stabilizer to help with foam stability. The stabilizer can be present in an amount of about 0-1%, or about 0-0.5%, or about 0.1-0.5%, based on the total weight of the rinse-free shampoo composition (i.e., including all three phases). In certain embodiments, the stabilizer can be phenoxyethanol.

In various embodiments, the rinse-free shampoo composition can include a plurality of components including, but not limited to, compounds for oil absorption and emollients. In some embodiments, the rinse-free shampoo composition can further include additional components such as fragrances, propellants, natural oil (e.g., coconut oil or argan oil), synthetic oil, and the like. A rinse-free shampoo composition of the present disclosure can comprise one or more of each type of component. However, each type of component is not necessarily present in different embodiments of the rinse-free shampoo compositions described herein.

In some embodiments, the rinse-free shampoo composition can comprise at least one surfactant useful for oil absorption. For example, in certain embodiments, the rinse-free shampoo composition can comprise cocamidopropyl betaine or any other amphoteric surfactant useful in removing oils from hair.

In some embodiments of the present disclosure, the weight percentage of the surfactant(s) in the rinse-free shampoo composition, based on the total weight of the rinse-free shampoo composition, can be 0.1% to about 10%, about 1% to about 5%, or about 2% to about 4. In certain embodiments, the weight percentage of surfactant(s) in the rinse-free shampoo composition, based on the total weight of the rinse-free shampoo composition, can be at least about 0.1%, at least about 1%, or at least about 3%, preferably with an upper limit of about 10% by weight.

In various embodiments of the present disclosure, the rinse-free shampoo composition can comprise an emollient useful for moisturizing the hair and thus acting as a conditioning agent. For example, in certain embodiments, the rinse-free shampoo composition can comprise PEG-8 dimethicone.

In some embodiments, the emollient particularly is a hydrophobic emollient. For example, the emollient of the rinse-free shampoo composition can comprise a long chain alkane. As used herein, a long chain alkane is at least a C₁₀ alkane or at least a C₁₂ alkane, preferably up to a C₄₀ alkane. For example, in certain embodiments the rinse-free shampoo composition can comprise an emollient in the form of a C₁₃-C₁₅ alkane. In some embodiments, the emollient comprises a squalane and/or one or more derivatives thereof, particularly hemisqualane.

In some embodiments, the rinse-free shampoo composition can comprise one or more additional conditioning agents known in the art, in addition to one or more hydrophobic emollients. Additional conditioning agents known in the art include, but are not limited to, silicones (e.g., phenyl trimethicones, dimethicones, cyclomethicones, dimethicone copolyols, amino silicones, etc.), petroleum based cationic surfactants, distearyldimonium chloride, guar compounds including cationic polymers and guar gum, polycationic compounds designated as polyquaternium 4, 6, 7, 10, or 22, etc. Some of the additional conditioning agents may provide other functions, such as being a solvent or a flow aid. Preferably, any such additional conditioning agents are present only in minor concentrations. In various embodiments of the present disclosure, additional conditioning agents can be expressly excluded from the rinse-free shampoo composition such that one or more hydrophobic emollients (e.g., one or more long chain alkanes) are the only conditioning agents present in the rinse-free shampoo composition. For example, in certain embodiments, the only conditioning agent present in the rinse-free shampoo composition is a hydrophobic emollient such as hemisqualane. In some embodiments, the rinse-free shampoo composition can be substantially free of additional conditioning agents beyond at least one hydrophobic emollient as described herein. As used herein, the term “substantially free of” means that the specified component is present in an amount of less than 0.1 percent by weight, based on the total weight of the composition. In some embodiments, the rinse-free shampoo composition can be entirely free of additional conditioning agents, meaning that not even trace amounts of additional conditioning agents are present in the rinse-free shampoo composition beyond the one or more hydrophobic emollients described herein—e.g., long chain alkanes. In certain embodiments, the rinse-free shampoo composition is substantially or entirely free of silicones and/or petroleum based cationic surfactants. In some embodiments, the rinse-free shampoo composition is substantially or entirely free of distearyldimonium chloride, for example.

In some embodiments of the present disclosure, the weight percentage of the emollient(s) in the rinse-free shampoo composition, based on the total weight of the rinse-free shampoo composition, can be 0.1% to about 15%, about 1% to about 12%, about 2% to about 10%, or about 2% to about 5%. In certain embodiments, the weight percentage of emollient(s) in the rinse-free shampoo composition, based on the total weight of the rinse-free shampoo composition, can be at least about 0.1%, at least about 1%, or at least about 3%, preferably with an upper limit of about 15% by weight.

In various embodiments of the present disclosure, the rinse-free shampoo composition can comprise PEG-40 hydrogenated castor oil. Without intending to be limited by theory, PEG-40 hydrogenated castor oil can be useful as an anti-caking agent. It is noted that PEG-40 hydrogenated castor oil can further function as a surfactant, a solubilizer, an emulsifier, an emollient, and/or a cleansing agent. The PEG-40 castor oil can be present in an amount of about 0.01 to about 1 weight percent, about 0.01 to about 0.5 weight percent, about 0.01 to about 0.3 weight percent, or about 0.02 to about 0.12 weight percent, based on the total weight of the rinse-free shampoo composition. In certain embodiments, the weight percentage of PEG-40 castor oil in the rinse-free shampoo composition, based on the total weight of the rinse-free shampoo composition, can be at least about 0.01%, at least about 0.1%, at least about 0.15%, or at least about 0.3%, preferably with an upper limit of about 5% by weight.

In various embodiments of the present disclosure, the rinse-free shampoo composition can comprise at least one fragrance. In some embodiments, a fragrance can be provided in the form of an essential oil. In certain embodiments, the weight percentage of one or more fragrances in the rinse-free shampoo composition, based on the total weight of the rinse-free shampoo composition, can be about 0.001% to about 3%, about 0.01% to about 2%, or about 0.1% to about 1%. In certain embodiments, the weight percentage of a fragrance in the rinse-free shampoo composition, based on the total weight of the rinse-free shampoo composition, can be at least about 0.001%, at least about 0.01%, at least about 0.1%, or at least about 1%, preferably with an upper limit of about 10% by weight.

In various embodiments of the present disclosure, the rinse-free shampoo composition can comprise at least one cooling agent (also referred to as a “refreshing agent”). In certain embodiments, the cooling agent can be in the form of an extract. For example, the rinse-free shampoo composition can include menthol. Without intending to be limited by theory, menthol can be used as a refresher or a cooling agent in the compositions disclosed herein such that the composition provides a cooling effect for the user upon application of the composition to the hair. The at least one extract (e.g., menthol) can be present in an amount of about 0.001 to about 1 weight percent, about 0.01 to about 0.5 weight percent, about 0.01 to about 0.3 weight percent, or about 0.02 to about 0.12 weight percent, based on the total weight of the rinse-free shampoo composition. In certain embodiments, the weight percentage of menthol in the rinse-free shampoo composition, based on the total weight of the rinse-free shampoo composition, can be at least about 0.001%, at least about 0.01%, at least about 0.1%, at least about 0.15%, or at least about 0.3%, preferably with an upper limit of about 5% by weight.

In various embodiments, the liquid phase of the rinse-free shampoo compositions can further comprise water in an amount such that the total weight percentage of the liquid phase of the rinse-free shampoo composition (i.e., including all other components of the liquid phase) is about 60-70% of the total weight of the rinse-free shampoo composition. Despite the fact the formulation has a high percentage of water (>20%) it leaves the hair feeling rinse-free after application. The viscosity of the liquid phase of the rinse-free composition can be in the range of about 1-10 centipoise, or about 2-8 centipoise. Without being limited by theory, it is believed that the ability to rinse-free quickly is due to the novel ratios between the ingredients and also the valve/actuator combination used to deliver it.

### Solid Phase

In various embodiments of the present disclosure, the rinse-free shampoo composition can comprise a cleaning agent useful for removing (e.g., absorbing) oil, grease, and other undesirable elements from hair. For example, the rinse-free shampoo composition can comprise at least one starch material that is effective to absorb at least a portion of any oils present on hair. Starch materials useful in embodiments of the rinse-free shampoo compositions described herein can also include any starch that is a thickener and/or has an emulsifying effect. Starch materials useful in the present disclosure include cornstarch, potato starch, tapioca starch, rice starch, wheat starch, cassaya starch, and combinations thereof. In certain embodiments, the rinse-free shampoo composition comprises rice starch. A starch material can be modified (e.g., through processes such as esterification, etherification, oxidation, acid hydrolysis, crosslinking, or enzyme conversion) or unmodified. In various embodiments, the rinse-free shampoo composition comprises distarch phosphate.

In some embodiments of the present disclosure, the weight percentage of starch material in the rinse-free shampoo composition, based on the total weight of the rinse-free shampoo composition, can be about 0.1% to about 15%, about 0.1% to about 10%, about 1% to about 8%, or about 4% to about 8%. In certain embodiments, the weight percentage of starch material in the rinse-free shampoo composition, based on the total weight of the rinse-free shampoo composition, can be at least about 0.1%, at least about 1%, at least about 4%, at least about 8%, at least about 10%, or at least about 15%, preferably with an upper range limit of about 50% by weight.

### Gas Phase

In some embodiments, a rinse-free shampoo composition is provided as an aerosol (e.g., an aerosol rinse-free shampoo composition) and can comprise at least one propellant. Non-limiting examples of propellants include butane, isobutane, propane, liquefied petroleum gas, dimethyl ether, trichlorofluoromethane, dichlorodifluoromethane, dichlorotetrafluorothane, monochlorodifluoromethane, trichlorotrifluoroethane propane, carbon dioxide, nitrous oxide, and combinations thereof. The term “aerosol rinse-free shampoo composition,” as used herein, refers to a composition comprising a rinse-free shampoo composition and a propellant. When a rinse-free shampoo composition is provided as an aerosol as compared to a powder, it may advantageously allow for the rinse-free shampoo composition to be applied in a diffusive manner, and may increase the transparency of the rinse-free shampoo composition when compared to powder application. After aerosol the aerosol rinse-free shampoo composition onto the hair, the propellant and the liquid phase of the rinse-free shampoo composition evaporates and a rinse-free powder remains.

In some embodiments of the present disclosure, the weight percentage of the propellant in the rinse-free shampoo composition, based on the total weight of the rinse-free shampoo composition, can be about 20% to about 35%, or about 25% to about 30%. In certain embodiments, the weight percentage of propellant in the rinse-free shampoo composition, based on the total weight of the rinse-free shampoo composition, can be at least about 20%, at least about 25%, or at least about 30%, preferably with an upper limit of about 35% by weight.

### Method of Forming a Rinse-Free Shampoo Composition and Application Thereof

A method of preparing a rinse-free shampoo product is also provided herein. In a main vessel, each ingredient in the liquid phase can be mixed together until fully blended. Any components of the solid phase can be separately mixed together until fully blended. The final product aerosol can then be filed with the appropriate levels of the liquid phase and the solid phase. The gas phase (i.e., propellant(s)) can be added to the aerosol can using a gassing hose.

Embodiments of the rinse-free shampoo compositions described herein are in the form of an aerosol which, when discharged, is in the form of a foam that can be applied to the hair of a user. The applied composition can be distributed through the hair by massaging into the hair, for example, in order to remove grease, oil and other undesirable elements from soiled hair. Any excess or residual rinse-free shampoo composition powder can be removed from the hair by combing or brushing the hair, for example. In certain embodiments, the rinse-free shampoo composition may leave little or no residue in the hair after application.

Upon dispensing, the rinse-free composition is in the form of a foam. The density of the foam dispensed from the product container can be in the range of about 0.05-0.25 g/cm³, or about 0.1-0.2 g/cm³.

The present rinse-free shampoo composition not only provides a cleansing effect but also is quick to use, and does not leave any residue in the hair after use. In certain embodiments, the foam/mousse provided by the rinse-free shampoo compositions described herein is packaged in any container known in the art that is capable of dispensing a foam. The container housing the rinse-free shampoo compositions described herein can be configured such that the rinse-free shampoo is delivered at a rate in the range of about 0.75-3.0 g/s, or about 1.0-2.8 g/s from the container.

### EXPERIMENTAL

### Example 1

A rinse-free shampoo according to the present disclosure was prepared. The composition is provided in Table 1 (with weight percentage ranges being based on the total weight of the respective rinse-free shampoo composition).

TABLE 1
Composition of the Rinse-free Shampoo
Mousse of the Present Disclosure

| | INCI | Weight Percentage |
|---|---|---|
| Phase | Name | Range |
| Liquid Phase | Water | 50-70 |
| | Alcohol Denat. | 1-15 |
| | Cocamidopropyl betaine | 0.01-5 |
| | PEG-8 Dimethicone | 0.01-2 |
| | Phenyloxyethanol | 0.01-1 |
| | Fragrance | 0.01-0.2 |
| | Extract | 0.01-0.2 |
| Solid Phase | Distarch Phosphate | 4-8 |
| Gas Phase | IsoButane | 4-10 |
| | Propane | 4-10 |
| | Butane | 10-20 |

When dispensed, the mousse according to the composition of Table 1 above had a density of about 0.1-0.2 g/cm³. When applied to rinse-free hair, the foam quickly goes from wet to rinse-free and does not leave any residue.

### Example 2

The dry coming force for hair treated with a foam according to Example 1 above versus the dry coming force of a control-treated tress was evaluated. The primary technical function of many conditioning products is to lubricate the hair surface, and, in doing so, facilitate manageability and provide detangling benefits and lower combing friction. A common and highly consumer-relevant approach for measuring this lubrication involves an instrumental combing experiment. Testing involves use of an Instron tensile tester to measure frictional forces while a hair tress is pulled through a comb

Eight (8) European Medium Brown hair tresses (supplied by International Hair Importers) (3.0 g, 8″ length, 1″ wide) were used per treatment group. Before treatment, tresses were bleached using standard bleaching procedure with 6% hydrogen peroxide solution at pH 10.2 and 40° C. for 40 min in total.

Tresses were standardized by treating all tresses with 15 wt. % sodium laureth sulfate (SLES), 30% the weight of the tress of 15% SLES was applied on wet hair, massaged for 30 seconds, and rinsed for 30 s (40 C, 1 GPM flow rate). The tresses were allowed to dry overnight at 60% relative humidity and ambient temperature.

Control tresses were SLES treated again (i.e., in addition to the standardization treatment) with 15 wt. % sodium laureth sulfate (SLES), 30% the weight of the tress of 15% SLES was applied on wet hair, massaged for 30 seconds, and rinsed for 30 s (40 C, 1 GPM flow rate). The tresses were allowed to dry at about 60% relative humidity and ambient temperature.

Sample tresses were treated with the Example rinse-free shampoo. The amount of Example rinse-free shampoo used for each tress was equal to 15% of the weight of each tress. The rinse-free shampoo was applied on dry hair, massaged into the hair for about 30 seconds (until dry), and then a comb was run through the dry, treated tress 5 times.

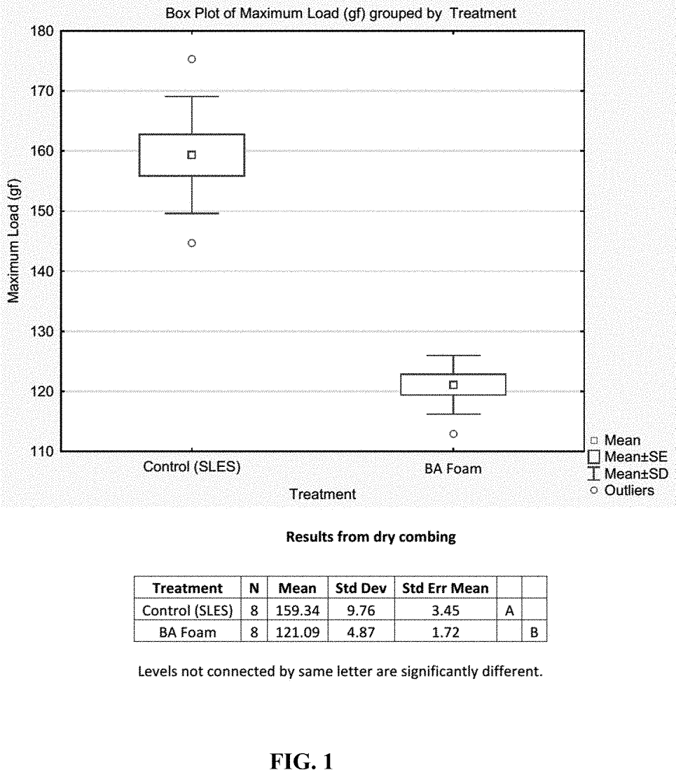

Dry combing measurements were performed. Six measurements were taken per tress using an Instron tensile tester to evaluate product performance. The tests were performed in accordance with the widely-used method first proposed by Garcia & Diaz (JSCC, 27, (1976) 379-398—Combability Measurements on Hair). Combing experiments were performed in the dry state after treatment. The results are shown in the box and whisker plots illustrated in FIG. 1, which were created using Statistica™; while JMP™ analytical software is used to perform the statistical analysis. Statistics are performed using the student's t-test at the 95% confidence level.

There is a statistically significant difference between tresses treated with the Example rinse-free shampoo-treated tresses and Control tresses (SLES). A decrease in dry combing force of approximately 24% is measured for the Example rinse-free shampoo-treated tresses when compared to Control (SLES-treated) tresses. % Reduction in combing forces is calculated according to the formula below:

$$\% Reduction in combing force = ( 1 - \frac{Combing force of treatment}{Combing force of control} ) \times 100$$

The calculated percent value as detailed above is based solely on the average mean for each treatment.

### Example 3

The performance of the rinse-free shampoo composition according to Example 1 above was evaluated for shine effect on treated hair. The increase in shine on sebum-treated hair as a result of applying the Example rinse-free shampoo was measured, as described below.

Eight (8) European Medium Brown hair tresses (supplied by International Hair Importers) (3.0 g, 8″ length, 1″ wide) were used per treatment group. Before treatment, tresses were bleached using standard bleaching procedure with 6% hydrogen peroxide solution at pH 10.2 and 40° C. for 40 min in total.

Tresses were standardized by treating all tresses with 15 wt. % sodium laureth sulfate (SLES), 30% the weight of the tress of 15% SLES was applied on wet hair, massaged for 30 seconds, and rinsed for 30 s (40 C, 1 GPM flow rate). The tresses were allowed to dry overnight at 60% relative humidity and ambient temperature.

Each of the tresses was treated with 1 g of sebum, which was applied and worked into each hair tress with a mascara brush (10 strokes per tress).

A group of the sebum-treated tresses was treated with a rinse-free shampoo composition according to Example 1 above. The amount of rinse-free shampoo used for each tress was equal to 15% of the weight of each tress. The rinse-free shampoo was applied on dry hair, massaged into the hair for about 30 seconds (until dry), and then a comb was run through the dry, treated tress 5 times. A control group of the sebum-treated tresses was not treated with the Example rinse-free shampoo. The control and rinse-free shampoo-treated tresses were allowed to dry overnight at 60% relative humidity and ambient temperature.

The following morning, a SAMBA device (a commercially-available device used to measure hair shine) was used to quantify shine on the hair tress samples. The SAMBA device operates in accordance with a collection of referenced literature articles (e.g., Bustard & Smith, Appl.Optics, 30, (1991), 3485; McMullen and Jachowicz, JSCC, 54, (2003), 335; McMullen and Jachowicz, JSCC, 55, (2004), 29), whereby the ratio of polarized and non-polarized light reaching the detector is used as an indicator of specular and diffuse reflection. These two values can then be employed in accordance with any of the equations in the scientific literature to produce numerical shine values. The intensity, breadth and contrast of the shine bands on the hair affect the impression of shine and are quantified as shine indices which are calculated from the measurement in the Samba device. These phenomena may be affected by hair color, reflectivity of the fibers' surface as well as the degree of alignment of the hair fibers. Without being limited by theory, deposition of materials on the highly reflective surface of hair should lead to a reduction in shine; yet consumers frequently ascribe benefits to commercial conditioning products. A plausible explanation involves the ability for conditioners to facilitate manageability, and by doing so, aid in the ability to induce hair fiber alignment. For this reason, a modified version of the tress holding device was designed and used, which allows hair to be combed into place.

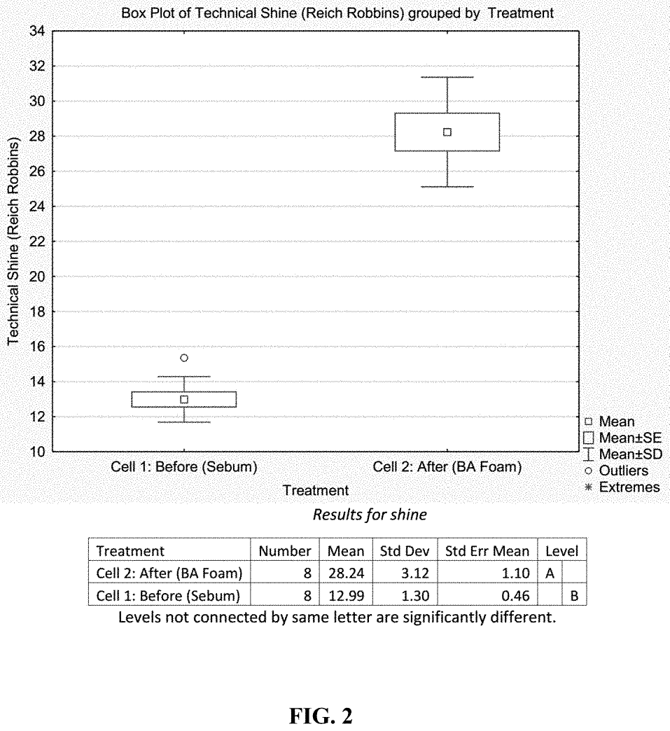

A box and whisker plot was generated (see, FIG. 2) using Statistica™, while JMP™ analytical software was used to calculate the statistics (student's t-test at 95% confidence level). The results show that hair treated with a rinse-free shampoo according to Example 1 above have a statistically significantly higher shine value than the Sebum treated cell.

### Example 4

The rinse-free shampoo composition according to Example 1 above was evaluated. ATR-FTIR spectroscopy was used to evaluate the efficiency of the Example rinse-free shampoo to clean hair tresses by removing sebum along hair fibers.

Caucasian medium brown hair tresses were used (supplied by International Hair Importers). Each tress was 8 inches long, 1 inch wide, and weighed approximately 3.0 g. Before treatment, tresses were bleached using standard bleaching procedure with 6% hydrogen peroxide solution at pH 10.2 and 40° C. for 40 min in total.

Tresses were standardized by treating all of the tresses with 0.15 ml of non-conditioning shampoo, massaging 30 s, and rinsing for 30 s (40 C, 1 GPM flow rate). The standardized tresses were left to equilibrate overnight at 60% relative humidity and ambient temperature prior to testing.

0.5 g of sebum was applied on standardized hair tresses to mimic greasy, dirty hair. The tresses were brushed for even distribution of the sebum along the hair fibers (10 brush strokes on front and back of the tress). The tresses were scanned by ATR-FTIR spectroscopy to assess the initial sebum content.

Sample tresses were treated (using gloved hands) with the Example rinse-free shampoo. The amount of Example rinse-free shampoo used for each tress was equal to 15% of the weight of each tress. The rinse-free shampoo was applied on dry hair, massaged into the hair for about 30 seconds (until dry). After 10 minutes, the hair tresses were brushed 10 times on each side. Two total applications were performed on the same hair tresses. After the second application, the tress was scanned by ATR-FTIR spectroscopy to evaluate the sebum removal.

The FTIR data were recorded with a spotlight system 400 from PerkinElmer with an ATR accessory. The spectra were recorded with the following spectral parameters:
Spectral resolution 4 cm⁻¹
8 Scans accumulations
Range 4000-650 cm⁻¹
   In the FTIR spectra, position and band intensity gives some information about the chemical nature of the material. For example, the contribution of esters always has a carbonyl (C═O) band around 1746 cm⁻¹. This band was used to evaluate the presence and the reduction of the sebum content on the hair tresses after application of Example rinse-free shampoo (according to Example 1 above).

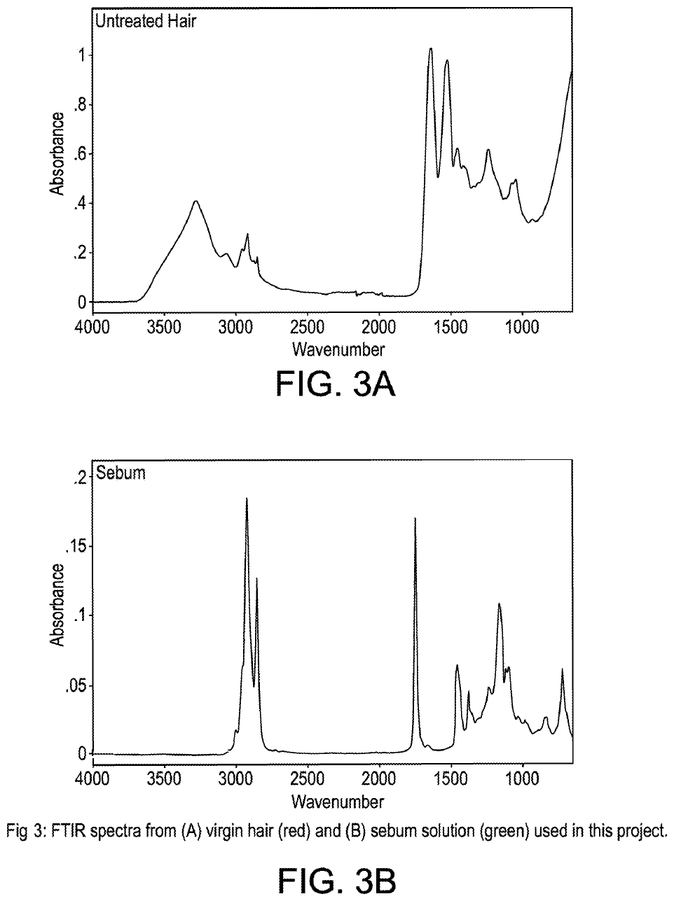

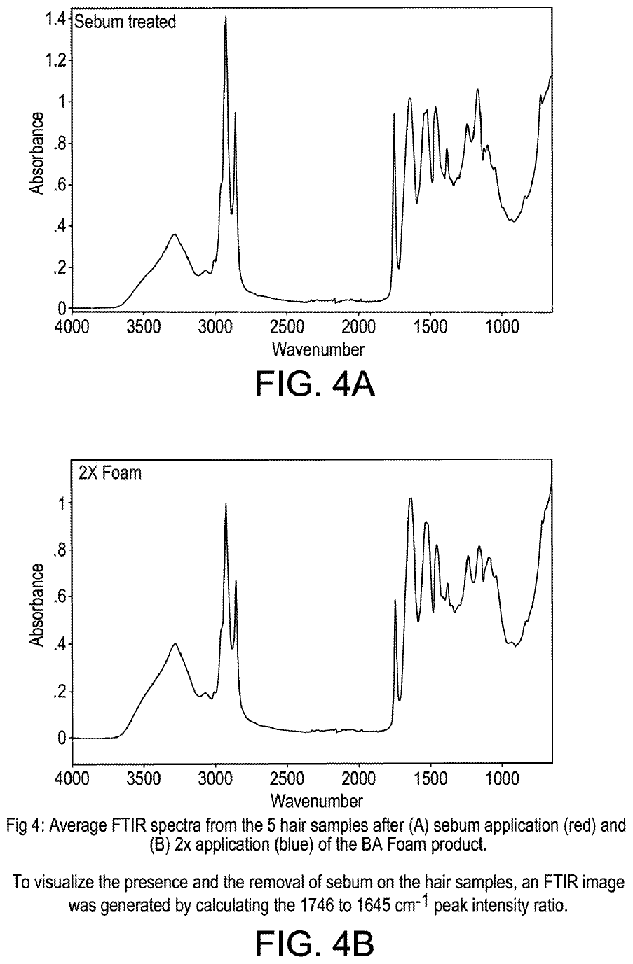

The FTIR spectrum from virgin hair and sebum solution used are shown in FIGS. 3A and 3B respectively. The hair samples were tested after sebum application as a positive control and after two product applications. To take into consideration the important variation inside the same hair tress, several FTIR spectra were recorded along various parts of the tress (root/middle/tip) for each measurement. A CO (sebum)/protein (Amide I) ratio was defined and calculated to assess the amount of sebum on the hair fibers. For each of the tresses, 12 spectra were recorded, baseline corrected, and averaged. The carbonyl (C═O) band around 1746 cm⁻¹ was used to follow the removal of sebum on the hair fibers after application of different dry shampoos (see, FIG. 4). FIG. 4A is a FTIR spectra for hair samples after sebum application. FIG. 4B is a FTIR spectra for hair samples after two times application of a dry shampoo composition according to the present disclosure. To visualize the presence and the removal of sebum on the hair samples, an FTIR image was generated by calculating the 1746 to 1645 cm⁻¹ peak intensity ratio.

After the application of the sebum onto the hair surface, a high and uniform sebum deposition was observed on each hair tresses. It is clearly seen that after two applications of the Example rinse-free shampoo, the sebum amount present on the hair surface significantly decreased. Accordingly, the rinse-free shampoo according to Example 1 above is effective at removing sebum from the hair. It is also noted that no visible residue was left on the hair tresses after product application.

### Example 5

The rinse-free shampoo composition according to Example 1 above was evaluated for protection against breakage.

Caucasian medium brown hair tresses were used (supplied by International Hair Importers). Each tress was 8 inches long, 1 inch wide, and weighed approximately 3.0 g. Before treatment, tresses were bleached using standard bleaching procedure with 6% hydrogen peroxide solution at pH 10.2 and 40° C. for 40 min in total. 10 tresses were used per treatment group.

Tresses were standardized by treating all tresses with 15 wt. % sodium laureth sulfate (SLES), 30% the weight of the tress of 15% SLES was applied on wet hair, massaged for 30 seconds, and rinsed for 30 s (40 C, 1 GPM flow rate). The tresses were allowed to dry at about 60% relative humidity and ambient temperature.

Control tresses were SLES treated again (i.e., in addition to the standardization treatment) with 15 wt. % sodium laureth sulfate (SLES), 30% the weight of the tress of 15% SLES was applied on wet hair, massaged for 30 seconds, and rinsed for 30 s (40 C, 1 GPM flow rate). The tresses were allowed to dry at about 60% relative humidity and ambient temperature.

Sample tresses were treated with the Example rinse-free shampoo. The amount of Example rinse-free shampoo used for each tress was equal to 15% of the weight of each tress. The rinse-free shampoo was applied on dry hair, massaged into the hair for about 30 seconds (until dry), and then a comb was run through the dry, treated tress 5 times.

A useful method to determine whether a treatment will protect hair against breakage is provided by repeated grooming experiments. In this test, the number of broken fibers is recorded as a function of repeated combing/brushing strokes. Any treatment that reduces snags, entanglements and abrasion can help in substantially lowering the number of broken fibers. A custom-built automated grooming device was used to comb the tresses.

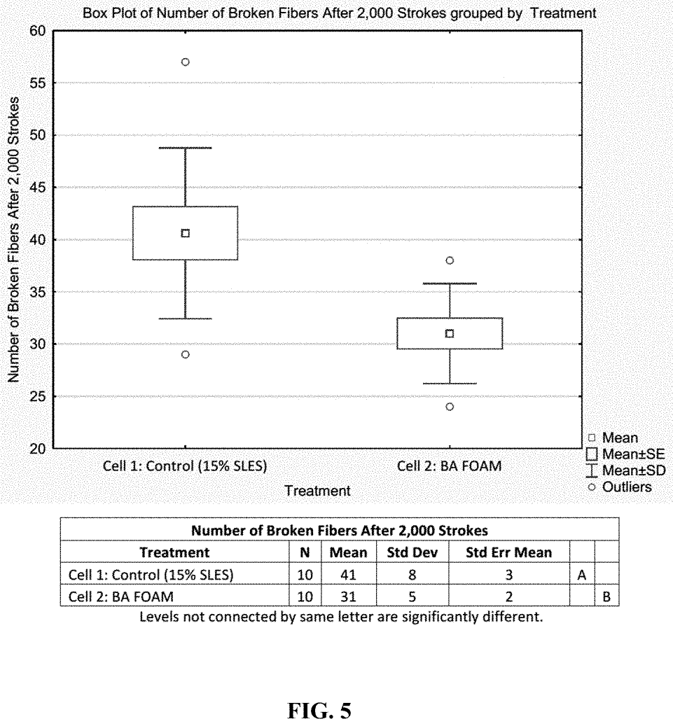

After the tresses were treated and allowed to equilibrate, the tresses were groomed using an automatic comber for a total of 2,000 cycles. Fibers were collected and assessed after every 200 cycles. Box and whisker plots were generated using Statistica™, while JMP™ analytical software was used to calculate the statistics (student's t-test at 95% confidence level). Results from repeated grooming testing after 2,000 brush strokes are shown in FIG. 5.

Hair treated with the Example rinse-free shampoo showed a statistically significant difference in breakage when compared to Control hair tresses (treated with 15% SLES only).

% Reduction in breakage is calculated as below:

$$\% Reduction in Breakage = ( 1 - \frac{Mean \# of Broken Fibers of treatment}{Mean \# of Broken Fibers of control} ) \times 100$$

Hair treated with the Example rinse-free shampoo showed a 24% decrease in breakage when compared to Control hair tresses (treated with 15% SLES only).

### Example 6

The rinse-free shampoo composition according to Example 1 above was subjectively evaluated by a group of 136 females, aged 18-55. The participants all wash their hair less often than daily and had a mix of hair types. Table 2 below provides a list of attributes provided by the rinse-free shampoo and the percentage of participants that gave positive endorsement of the attribute.

TABLE 2
Subjective Evaluation of Rinse-Free Shampoo

| Product Attribute | % Endorsement |
|---|---|
| The product absorbs quickly into hair | 82% |
| The product does not weight hair down | 82% |
| The product makes my hair feel fresh | 78% |
| The product leaves hair dry to the touch in 60 seconds | 76% |
| The product leaves no residue | 76% |
| The product leaves hair easy to manage | 74% |
| The product makes my hair feel soft | 74% |
| The product makes my hair feel refreshed | 74% |
| The product leaves hair feeling fresh | 73% |
| The product makes my hair feel clean | 68% |
| The product makes my hair feel nourished | 68% |
| The product gives freshly cleansed hair without stepping | 67% |
| in the shower | |
| The product gives fresh, healthy hair really fast | 66% |
| The product makes my hair feel clean instantly | 66% |
| The product makes my hair feel hydrated | 66% |

### Example 7

A rinse-free shampoo according to the present disclosure was prepared. The composition is provided in Table 3 (with weight percentage ranges being based on the total weight of the respective rinse-free shampoo composition).

TABLE 3
Composition of the Rinse-free Shampoo
Mousse of the Present Disclosure

| | INCI | Weight Percentage |
|---|---|---|
| Phase | Name | Range |
| Liquid Phase | Water | 50-70 |
| | Alcohol Denat. | 1-15 |
| | Cocamidopropyl betaine | 0.01-5 |
| | PEG-8 Dimethicone | 0.01-2 |
| | Phenyloxyethanol | 0.01-1 |
| | PEG-40 Hydrogenated Castor Oil | 0.01-0.3 |
| | Fragrance | 0.01-0.2 |
| | Extract (Menthol) | 0.001-0.2 |
| Solid Phase | Distarch Phosphate | 4-8 |
| Gas Phase | IsoButane | 4-10 |
| | Propane | 4-10 |
| | Butane | 10-20 |

When dispensed, the mousse according to the composition of Table 3 above had a density of about 0.1-0.2 g/cm³. When applied to rinse-free hair, the foam quickly goes from wet to rinse-free and does not leave any residue.

Many modifications and other embodiments of the disclosure will come to mind to one skilled in the art to which this disclosure pertains having the benefit of the teachings presented in the foregoing description; and it will be apparent to those skilled in the art that variations and modifications of the present disclosure can be made without departing from the scope or spirit of the disclosure. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A rinse-free shampoo composition, comprising:
an aqueous liquid phase, wherein the liquid phase is about 60-70% of the total weight of the rinse-free shampoo composition;
a solid phase comprising a starch thickener, wherein the solid phase is about 4-15% of the total weight of the rinse-free shampoo composition; and
a gas phase comprising at least one propellant, wherein the gas phase is about 20-35% of the total weight of the rinse-free shampoo composition.

2. The rinse-free shampoo composition of claim 1, wherein the liquid phase comprises an alcohol in the amount of about 1-15%, based on the total weight of the rinse-free shampoo composition.

3. The rinse-free shampoo composition of claim 2, wherein the alcohol is ethanol.

4. The rinse-free shampoo composition of claim 1, wherein the liquid phase comprises stabilizer in the amount of about 0.01-1%, based on the total weight of the rinse-free shampoo composition.

5. The rinse-free shampoo composition of claim 4, wherein the stabilizer is phenoxyethanol.

6. The rinse-free shampoo composition of claim 1, wherein the liquid phase comprises a conditioning agent in the amount of about 0.01-5%, based on the total weight of the rinse-free shampoo composition.

7. The rinse-free shampoo composition of claim 6, wherein the conditioning agent is PEG-8 dimethicone.

8. The rinse-free shampoo composition of claim 1, wherein the liquid phase comprises an amphoteric surfactant in the amount of about 0.01-3%, based on the total weight of the rinse-free shampoo composition.

9. The rinse-free shampoo composition of claim 8, wherein the amphoteric surfactant is cocamidopropyl betaine.

10. The rinse-free shampoo composition of claim 1, further comprising at least one of a fragrance, extracts, and preservatives.

11. The rinse-free shampoo composition of claim 1, wherein the liquid phase further comprises a cooling agent present in an amount of about 0.001 to about 1 weight percent, based on the total weight of the rinse-free shampoo composition.

12. The rinse-free shampoo composition of claim 11, wherein the cooling agent is menthol.

13. The rinse-free shampoo composition of claim 1, wherein the liquid phase further comprises PEG-40 hydrogenated castor oil in an amount of about 0.01 to about 1 weight percent, based on the total weight of the rinse-free shampoo composition.

14. The rinse-free shampoo composition of claim 1, wherein the starch thickener is distarch phosphate.

15. The rinse-free shampoo composition of claim 1, wherein the at least one propellant comprises isobutane, propane, butane, or a combination thereof.

16. The rinse-free shampoo composition of claim 1, wherein the liquid phase has a viscosity of about 1-10 centipoise.

17. The rinse-free shampoo composition of claim 1, wherein the rinse-free shampoo composition is provided in a container from which the rinse-free shampoo composition is dispensable in the form of a foam, wherein the foam has a density of about 0.1-0.2 g/cm³.

18. A method of cleaning hair comprising applying the rinse-free shampoo composition of claim 1 onto the hair of a human or animal.

18. od of claim 18, wherein the rinse-free shampoo is delivered at a rate of 0.75-3.0 g/s to the hair.

20. A device containing the rinse-free shampoo composition of claim 1.
